(19) United States
(12) Patent Application Publication
(10) Pub. No.: US 2007/0239147 A1
(43) Pub. Date: Oct. 11, 2007

(54) METHOD, SYSTEM AND APPARATUS FOR DERMATOLOGICAL TREATMENT AND FRACTIONAL SKIN RESURFACING

(75) Inventors: Dieter Manstein, Boston, MA (US); R. Rox Anderson, Boston, MA (US); Hans-Joachim Laubach, Neuerburg (DE); Tomi L. Wall, Walnut Creek, CA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 11/733,159

(22) Filed: Apr. 9, 2007

Related U.S. Application Data
(60) Provisional application No. 60/790,171, filed on Apr. 7, 2006.

(51) Int. Cl. A61B 18/18

(52) U.S. Cl. 606/9; 606/10

(57) ABSTRACT

A system, method and apparatus are provided for treating dermatological conditions by generating a relatively large amount of thermal damage beneath a skin surface while generating relatively little thermal damage to the skin surface and/or epidermis. For example, the exemplary system, method and apparatus provide for directing a radiation beam onto a target location on the skin surface to thermally damage a volume of tissue beneath the target location, and directing a second radiation beam onto the same target location to thermally damage a different volume of tissue beneath the target location. The radiation beams may include ablative and/or non-ablative laser beams, or they may be generated by, e.g., a flashlamp, a tungsten lamp, a diode, or a diode array. The two beams may be pulsed. Further, they can be part of a single continuous wave beam, which can be rotated around an axis which passes through the target area, or which can be directed at a plurality of angles with respect to the skin surface.

### CROSS-REFERENCE TO RELATED APPLICATION

This application is based upon and claims the benefit of priority from U.S. Patent Application Ser. No. 60/790,171, filed Apr. 7, 2006, the entire disclosure of which is incorporated herein by reference.

### FIELD OF THE INVENTION

The present invention relates to methods and apparatus that use electromagnetic radiation for dermatological treatment and, more particularly to methods, systems and apparatus that use optical radiation to ablate or thermally damage target sites of skin tissue below the skin surface for dermatological treatment while ablating or damaging a relatively small area of the epidermis.

### BACKGROUND INFORMATION

There is an increasing demand for repair of or improvement to skin defects, which can be induced by aging, sun exposure, dermatological diseases, traumatic effects, and the like. Certain treatments which use electromagnetic radiation have been used to improve skin defects by inducing a thermal injury to the skin, which results in a complex wound healing response of the skin. This leads to a biological repair of the injured skin.

Various techniques providing this objective have been recently described in the literature. These different techniques can be generally categorized in three groups of treatment modalities, such as, e.g., ablative laser skin resurfacing (“LSR”), non-ablative collagen remodeling (“NCR”), and fractional resurfacing (“FS”). The first group of treatment modalities, i.e., LSR, includes techniques for causing thermal damage to the epidermis and/or dermis, while the second group, i.e., NCR, includes procedures to spare thermal damage of the epidermis. The latter group, i.e., FS, includes techniques that involve inducing thermal damage in small-scale regions of epidermal and/or dermal tissue, while leaving adjacent regions undamaged to promote beneficial healing processes and reduce scarring.

The use of LSR with pulsed CO₂ or Er:YAG lasers, which may be referred to in the art as laser resurfacing or ablative resurfacing, can be an effective treatment option for signs of photo aged skin, chronically aged skin, scars, superficial pigmented lesions, stretch marks, and superficial skin lesions.

LSR treatments can result in thermal damage and/or ablative removal of the upper skin surface of the area being treated, including the epidermis and optionally portions of the upper dermis. The LSR treatment with pulsed CO₂ lasers can be particularly aggressive, likely causing a thermal skin damage to the epidermis and at least to the superficial dermis. Following the LSR treatment using CO₂ lasers, a high incidence of complications can occur, including persistent erythema, hyperpigmentation, hypopigmentation, scarring, and infection (e.g., infection with Herpes simplex virus). The LSR treatment with the Er:YAG laser has been discussed as a less destructive and lower pain alternative to the CO₂ laser, due to the lesser penetration depth of the Er:YAG pulsed laser. Using the Er:YAG laser can result in a thinner zone of thermal injury within the residual tissue of the target area of the skin. However, LSR that uses the Er:YAG laser may produce side effects similar to those made by the LSR procedure that uses the CO₂ laser within the first days after the treatment.

Patients may experience major drawbacks after each LSR treatment, including edema, oozing, and burning discomfort during, e.g., first fourteen (14) days after treatment. These drawbacks can be unacceptable for many patients. A further problem with the LSR procedures may be that the procedures are relatively painful, and therefore may generally require an application of a significant amount of analgesia. While the LSR procedure for relatively small areas can be performed under local anesthesia provided by injection of an anestheticum, generally, the LSR procedures for relatively large areas may be frequently performed under general anesthesia or after nerve blockade by multiple injections of anesthetic.

One of the limitations of the LSR procedures that use CO₂ or Er:YAG lasers is that ablative laser resurfacing generally may not be effectively performed on the patients with dark complexions. For example, the removal of a pigmented epidermis tissue can cause a severe cosmetic disfigurement to patients with a dark complexion. Such problem may last from several weeks up to years, which is considered by some patients and physicians to be unacceptable. Another limitation of the LSR procedures is that ablative resurfacing in areas other than the face generally have a greater risk of scarring. The LSR procedures in areas other than the face result in an increased incidence of an unacceptable scar formation because the recovery from skin injury within these areas may not be very effective.

In an attempt to overcome such problems associated with the LSR procedures, NCR techniques have been developed. These techniques are variously referred to in the art as non-ablative resurfacing, non-ablative subsurfacing, or non-ablative skin remodeling techniques. NCR techniques can generally utilize non-ablative lasers, flashlamps, or radio frequency current to damage dermal tissue while sparing damage to the epidermal tissue. The NCR techniques generate the thermal damage of only the dermal tissues, which are believed to induce wound healing which results in a biological repair and a formation of new dermal collagen. This type of wound healing can result in a decrease of photoaging related structural damage. By avoiding epidermal damage in NCR techniques, the severity and duration of treatment related side effects can be decreased. In particular, post procedural oozing, crusting, pigmentary changes and incidence of infections due to prolonged loss of the epidermal barrier function can usually be avoided by using the NCR techniques.

Various strategies are presently applied using nonablative lasers to achieve damage to the dermis while sparing the epidermis. The nonablative lasers used in the NCR procedures have a deeper dermal penetration depth as compared to ablative lasers used in LSR procedures. Wavelengths in the near infrared spectrum can be used. These wavelengths can generally cause the non-ablative laser to have a deeper penetration depth than the very superficially-absorbed ablative Er:YAG and CO₂ lasers. The dermal damage is achieved by a combination of proper wavelength and superficial skin cooling, or by focusing a laser into the dermis with a high numerical aperture optic in combination with superficial skin cooling. While that these techniques can assist in avoiding epidermal damage, one of the drawbacks of such techniques is their limited efficacies. An improvement of photoaged skin or scars after the treatment with the NCR techniques is significantly smaller than the improvements obtained when the LSR ablative techniques are utilized. Even after multiple treatments, the clinical improvement is often far below the patient's expectations. In addition, a clinical improvement is usually several months delayed after a series of treatment procedures.

Another limitation of the NCR procedures relates to the breadth of acceptable treatment parameters for safe and effective treatment of dermatological disorders. The NCR procedures generally rely on an optimum coordination of laser energy and cooling parameters, which can result in an unwanted temperature profile within the skin leading to either no therapeutic effect or scar formation due to the overheating of a relatively large volume of the tissue.

Yet another disadvantage of the non-ablative procedures relates to the sparing of the epidermis. While sparing the epidermis is advantageous to decrease the side effects related to complete removal of the epidermis, several applications of NCR procedures may benefit from at least a partial removal of the epidermal structures. For example, photoinduced skin aging manifests not only by dermal alterations, but also by epidermal alterations.

A further disadvantage of both ablative and nonablative resurfacing is that the role of keratinocytes in the wound healing response is not capitalized upon. Keratinocyte generally plays an active role in the wound healing response by releasing cytokines when the keratinocyte is damaged. During traditional ablative resurfacing procedures, the keratinocytes are removed from the skin along with the epidermis, thereby completely removing them from the healing process. On the other hand, in the traditional non-ablative procedures, the keratinocytes (which are located in the epidermis) are not damaged, therefore they do not release cytokines to aid in the healing process.

Still another drawback with all LSR and NCR techniques now used is the appearance of visible spots and/or edges after treatment due to inflammation, pigmentation, or texture changes, corresponding to the sites of treatment. Devices implementing the LSR and NCR procedures produce macroscopic (easily seen) exposure areas. For example, laser exposure spot diameters typically vary from about 1 to 10 mm, and NCR exposure spot diameters from about 3 to 50 mm. Some devices, such as intense pulsed light devices, leave “boxes” of skin response due to rectangular output patterns on the skin. Patients are not pleased about such spot or box patterns, easily seen as red, brown or white areas ranging from on the order of millimeters to centimeters in size, which remain for days or even years after treatment.

To address the problems associated with both LSR and NCR techniques, a fractional resurfacing (FS) approach has been developed. For example, fractional resurfacing techniques generally involve the controlled ablation, removal, destruction, damage or stimulation of multiple small (generally less than 1 mm) individual exposure areas of skin tissue with intervening spared areas of skin tissue, performed as a treatment to improve the skin. The spared tissue accelerates the healing process of the damaged regions. Because certain portions of the target area remain undamaged, the FS techniques can take advantage of thereby preserving keratinocytes and melanocytes, which serve as a pool of undamaged cells to promote reepithelialization. This procedure differs from the traditional resurfacing procedures, in that the entirety of the target area (or at least most of it) is damaged.

In treatments that use the FS procedures, the individual exposure areas may be oval, circular, arced and/or linear in shape. Fractional resurfacing damage patterns may also be formed as adjacent, often parallel lines of damage over the skin surface that extend to a certain depth into the dermal tissue. Such FS patterns can be formed by, e.g., translating one or more point sources of optical energy over the surface of the area to be treated, which can result in one or more lines of damaged tissue extending from the skin surface downward.

The spatial scale of fractional resurfacing can be selected to avoid the appearance of various spots or boxes on a macroscopic scale, while still providing effective treatment because the multiple small areas can be exposed to greater than a minimal stimulus. For example, removal or photothermal destruction of thousands of 0.1 mm diameter individual exposure areas, spaced 0.2 mm apart, and extending into the skin up to a depth of 0.5 mm, is well tolerated, and can produce effective improvement of photoaging, without apparent spots and with rapid healing. Spared skin between the individual exposure areas rapidly initiates a wound healing response, which is better tolerated than conventional LSR procedures.

It may be desirable to produce extensive thermal damage or ablation below the skin surface, particularly in the dermis region, to increase the degree of a stimulated healing response. Such damage can induce a greater degree of beneficial wound healing effects, such as improvement in pigmentation appearance including, e.g., tattoo removal, and/or greater skin tightening arising from increased collagen denaturation. However, conventional FS techniques are limited in the amount of thermal damage or ablation that can be created below the epidermis by the need to avoid ablating or damaging too much of the epidermal tissue.

Therefore, there may be a need to provide systems, processes and apparatus which combine safe and effective treatment for improvement of dermatological disorders with minimum side effects, such as intra procedural discomfort, post procedural discomfort, lengthy healing time, and post procedural infection. Further, there may be a need to provide a system, process and apparatus which are capable of inducing greater amounts of thermal damage below the surface of the skin, while avoiding excessive damage to the epidermis. Another object of the present invention is to overcome or at least reduce some of the above-described deficiencies.

### SUMMARY OF THE INVENTION

It is therefore one of the objects of the present invention to provide a system, process and apparatus which combine safe and effective treatment to improve dermatological disorders with fewer side effects. Another object of the present invention is to provide a system, process and apparatus which cause a smaller amount of damage to the epidermis while inducing a greater degree of well-controlled thermal damage below the skin surface.

These and other objects can be achieved with the exemplary embodiments of the apparatus and method according to the present invention, in which one or more radiation sources can be configured to induce thermal damage and/or ablation to a plurality of regions of below the surface of the skin, each of the regions extending into the skin tissue from a common target site on the surface.

In another exemplary embodiment of the present invention, a source of electromagnetic radiation can be directed by an optical arrangement to generate one or more patterns of thermal damage or ablation below the skin, where each pattern can originate at a single target site on the surface of the skin.

The radiation sources can include, e.g., an ablative laser such as, e.g., a CO2 laser or an ER:YAG laser, a non-ablative laser, a flashlamp, a tungsten lamp, a diode, or a diode array. The optical arrangement can include a beam splitter and/or one or more mirrors which may be movable with respect to the radiation sources.

A vacuum arrangement can be provided which is configured to pull the portion of the skin surface containing the target location closer to the optical arrangement. This can stretch the skin in the vicinity of the target location, which may allow more accurate positioning of the radiation with respect to the skin. The vacuum arrangement may also promote more rapid healing of the damaged area, especially near or at the target area, by providing relative compression of the area after the vacuum is released and the skin is no longer being stretched.

In certain exemplary embodiments of the present invention, the radiation may be provided in a form of one or more pulses. Alternatively, the radiation may be provided in a form of a continuous wave beam of energy.

In yet another exemplary embodiment of the present invention, a source of electromagnetic radiation can be directed by an optical arrangement to generate one or more patterns of thermal damage or ablation below the skin, where some or all of the patterns may have the form of one or more lines that form an acute angle with respect to the plane of the skin surface, and which intersect the skin surface at a common target site. The individual patterns may be formed using different angles with respect to the skin surface.

In certain exemplary embodiments of the present invention, the pattern may have a form of the surface of a cone formed by rotating a beam around an axis that passes through the target site. The vertex of such cone can correspond to the target site located at the surface of the skin. In a still further exemplary embodiment of the present invention, several patterns may be combined with each intersecting the skin surface at a single common target site.

In a further exemplary embodiment of the present invention, a plurality of target sites on the skin surface may be treated, with one or more patterns of thermal damage and/or ablation originating at each target site and extending below the surface.

These and other objects, features and advantages of the present invention will become apparent upon reading the following detailed description of embodiments of the invention, when taken in conjunction with the included drawings.

### BRIEF DESCRIPTION OF THE DRAWINGS

For a more complete understanding of the present invention and its advantages, reference is now made to the following description, taken in conjunction with the accompanying drawings, in which:

FIG. 1 is a cross-section view of an exemplary damage pattern that may be produced using conventional fractional resurfacing techniques;

FIG. 2 is an illustration of an exemplary apparatus/system that may be used in accordance with exemplary embodiments of the present invention;

FIG. 3A is a cross-sectional view of exemplary damage patterns that may be produced in accordance with the system, process and apparatus of the exemplary embodiments of the present invention;

FIG. 3B a top view of the exemplary damage patterns illustrated in FIG. 3A;

FIG. 4A is a cross-sectional view of further exemplary damage patterns that may be produced in accordance with the system, process and apparatus of the exemplary embodiments of the present invention;

FIG. 4B is a top view of the exemplary damage patterns illustrated in FIG. 4A;

FIG. 5 is a perspective cross-sectional view of an additional exemplary damage pattern that may be produced in accordance with the system, process and apparatus of the exemplary embodiment of the present invention;

FIG. 6 is an illustration of an exemplary optical system/arrangement/apparatus that may be used in accordance with certain exemplary embodiments of the present invention; and

FIG. 7 is an illustration of a side view of an exemplary embodiment of a vacuum apparatus that may be used in accordance with exemplary embodiments of the present invention.

Throughout the drawings, the same reference numerals and characters, unless otherwise stated, are used to denote like features, elements, components, or portions of the illustrated embodiments. Moreover, while the present invention will now be described in detail with reference to the Figures, it is done so in connection with the illustrative embodiments.

### DETAILED DESCRIPTION OF THE PREFERRED EMBODIMENTS

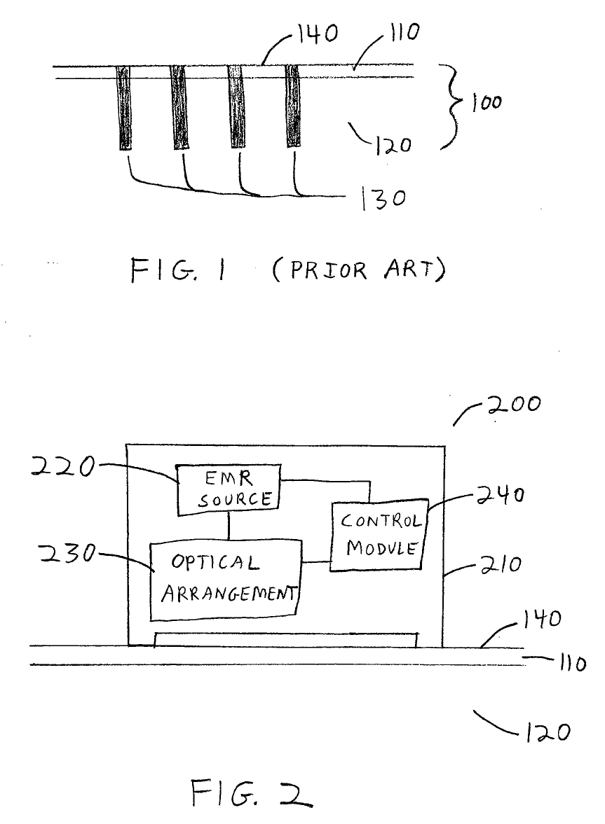

FIG. 1 shows a cross-sectional illustration of exemplary damage patterns produced by conventional fractional resurfacing of a target area of skin. An area of skin 100 includes an upper epidermis layer 110, and a lower dermis layer 120. A number of regions of thermal damage 130 extend from the surface of the skin 140 through the epidermis 110 and into the dermis 120.

These regions of thermal damage 130 can include tissue that has been heated sufficiently to cause at least some degree of tissue damage and/or cell death. The thermal damage can be produced by exposing the skin to a number of collimated beams generated by a source of electromagnetic radiation (“EMR”). These beams can be generated, e.g., by lasers, diodes, diode arrays, etc. Optionally, a portion of the regions of thermal damage 130 can include locations where the tissue has been ablated. Such ablation can be achieved, for example, by the use of highly-absorbed and/or high-powered EMR sources such as, e.g., CO2 lasers or erbium-yttrium-aluminum-garnet (“Er:YAG”) lasers.

The regions of thermal damage 130 may have the shape of thin cylindrical or ellipsoidal columns or, alternatively, thin planes perpendicular to the plane of FIG. 1. Such planes may appear as a series of lines when viewed from above the surface of the skin 140, and can be formed by translating an array of point sources along the surface of the skin 140. The width, depth and shape of the damaged regions 130 can be controlled by specifying, for example, the type of EMR source used, the power supplied to the EMR source, whether the EMR beams are pulsed or continuous, the duration of the pulses, the details of an optical arrangement used to direct the EMR beams from the EMR source into the skin, etc. Methods and apparatus for performing fractional resurfacing techniques are described, e.g., in International Publication No. WO 2004/086947.

FIG. 2 illustrates an exemplary system/apparatus 200 that may be used for a dermatological treatment in accordance with an exemplary embodiment of the present invention. The exemplary system/apparatus 200 includes a housing 210 that can be positioned in contact with the surface of the skin 140 over an area to be treated. The apparatus also can include an EMR source 220, an optical arrangement 230, and a control module 240. These exemplary components may be located within the housing 210 as illustrated in FIG. 2, or part or all of any of such components may be located outside the housing 210. The control module 240 can be in communication with the EMR source 220, which in turn is operatively connected to the optical arrangement 230. The control module 240 can also be in communication with the optical arrangement 230.

In one exemplary embodiment of the present invention, the control module 240 can be in wireless communication with the EMR source 220. In another exemplary embodiment, the control module 240 may be in wired communication with the EMR source 220. In another exemplary embodiment of the present invention, the control module 240 can be located outside of the housing 210. In yet another exemplary embodiment, the EMR source 220 can be located outside of the housing 210. In a further exemplary embodiment, the control module 240 and the EMR source 220 may each located outside of the housing 210.

The control module 240 can provide application specific settings to the EMR source 220. The EMR source 220 can receive these settings, and generate EMR based on these settings. The settings can control, for example, the wavelength of the EMR, the energy delivered to the skin, the power delivered to the skin, the pulse duration for each EMR pulse, the fluence of the EMR delivered to the skin, the number of EMR pulses, the delay between individual EMR pulses, the beam profile of the EMR, etc. The energy produced by the EMR source 220 can be an optically-generated radiation, which may be focused, collimated and/or directed at least in part by the optical arrangement 230 toward the surface of the skin 140.

In one exemplary embodiment of the present invention, the EMR source 220 may be a laser, a flashlamp, a tungsten lamp, a diode, a diode array, and the like. In another exemplary embodiment of the present invention, the EMR source 220 can be a CO₂ laser or an Er:YAG laser.

Prior to being used in a dermatological treatment, the system/apparatus 200 shown in FIG. 2 can be configured by a user. For example, the user may interface with the control module 240 in order to specify certain settings usable for a particular procedure. Further, the user may specify the wavelength of the EMR, the energy delivered to the skin, the power delivered to the skin, the pulse duration for each EMR pulse, the fluence of the EMR delivered to the skin, the number of EMR pulses, the delay between individual EMR pulses, the beam profile of the EMR, the number and direction of pulses applied to a single target site, etc. The EMR source 220 may be configured to produce a collimated pulsed EMR irradiation with a wavelength ranging from about 400 to 11,000 nm, and preferably near 3.0 μm when using an Er:YAG, laser and near about 10.6 μm when using a CO₂ laser as the EMR source.

The collimated pulsed EMR irradiation may be applied which has a pulse duration in the range of about 1 μs to 10 s, preferably in the range of about 100 μs to 100 ms, and more preferably in the range of about 0.1 ms to 10 ms, and fluence in the range from about 0.01 to 100 J/cm², and preferably in the range from about 1 to 10 J/cm². The applied EMR should be able to achieve a temperature rise at least within the exposed areas of the skin that is sufficient to cause thermal damage to the epidermis 110 and/or the dermis 120. The peak temperature sufficient to cause the thermal damage in the exposed tissues may be time dependent, and can be at least in the range of about 45° C. to 100° C. For exposure times in the range of about 0.1 ms to 10 ms, the minimum temperature rise that is likely to cause the thermal damage may be in the range of approximately 60° C. to 100° C. The depth and degree of thermal damage can be adjusted by a selection of wavelength, fluence per pulse, number of pulses, and/or direction of the pulses directed at each target site. Using such selected parameters, an ablation of some portion of the epidermal and/or dermal tissue can be achieved. Alternatively, the ablation can be avoided by selecting appropriate EMR source parameters that may include a lower fluence, a smaller amount of delivered energy, a shorter pulse duration, etc.

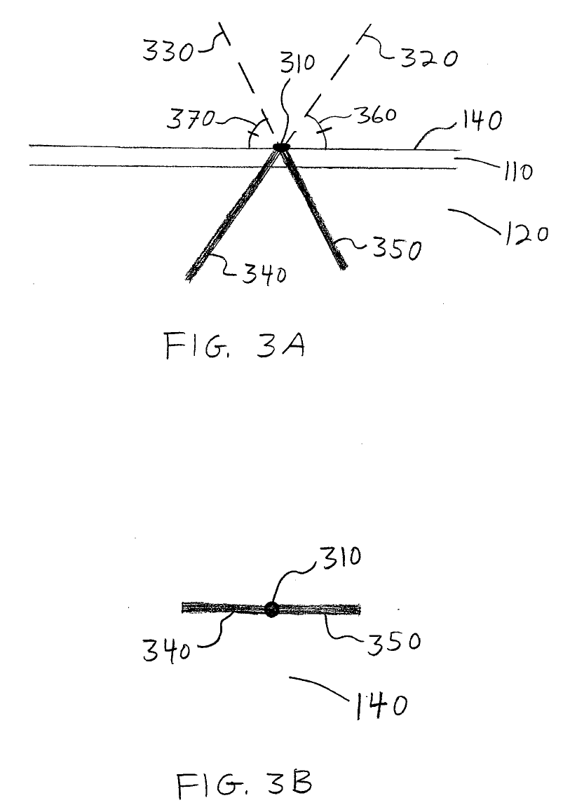

FIG. 3A illustrates a cross-sectional view of an exemplary damage pattern produced using the system process and apparatus according to an exemplary embodiment of the present invention. For example, two EMR beams 320, 330 can be directed at a common target site 310 located on the surface of the skin 140. These beams 320, 330 may form acute angles 360, 370, respectively, with respect to the skin surface 140. Thermal damage regions 340, 350 are formed within the skin by the EMR beams 320, 330. These damage regions 340, 350 may include the tissue that has been thermally damaged and/or heated sufficiently to be at least partly ablated.

FIG. 3B illustrates a top view of the thermal damage pattern illustrated in FIG. 3A. As described above, the damage regions 340, 350 extend below the surface of the skin 140, and away from the common target site 310. The damage regions 340, 350 may be parallel to one another as viewed from above and as shown in this figure, and/or they may form any angle with respect to each other as viewed from above.

Each target site 310 at the surface of the skin 140 shown in FIG. 3A has two damage regions 340, 350 associated therewith, as compared to each damage region 130 produced by the conventional fractional resurfacing technique that intersects the skin surface 140 as shown in FIG. 1. By using the exemplary system, process and apparatus in accordance with the exemplary embodiments of the present invention, it is possible to generate large volumes of thermal damage beneath the skin 140 while damaging the same amount of epidermal tissue at the surface of the skin 140 as compared with the damage caused by the conventional fractional resurfacing techniques.

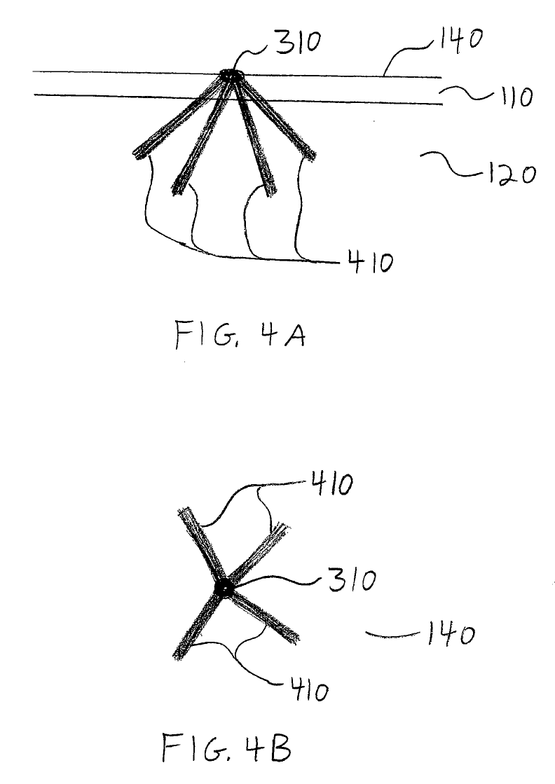

FIG. 4A illustrates a cross-sectional view of another exemplary damage pattern in which several damage regions 410 beneath the skin may be generated at each surface target site 310, in accordance with a further exemplary embodiment of the present invention. For example, each of these damage regions 410 intersects the surface of the skin 140 at the common target site 310. These damage regions 410 may form similar or varying angles with respect to the surface of the skin 140. The depth and/or width of these damage regions 410 may also be approximately equal to one another, and/or one or more may be varied by varying the parameters of the EMR beam that is used to form them.

FIG. 4B illustrates a top view of the thermal damage pattern illustrated in FIG. 4A. For example, each of the damage regions 410 shown in FIG. 4B extends below the surface of the skin 140 and away from a common target site 310. The damage regions 410 may form any angle with respect to each other as viewed from above.

The parameters which can be used to generate the damage patterns 410 associated with the target site 310 can be varied, depending on the desired effect to be produced. For example, the desired effect may depend on the dermatological treatment being administered, whether the thermal damage is to be ablative or involve mere heating of the skin tissue, the location and characteristics of any targeted structures within the skin tissue to be treated (including the depth), the characteristics of the skin being treated, etc. The parameters that may be varied to achieve the desired effects may include the various characteristics of the EMR source that can govern the diameter and length of the individual regions of damage, the number of damage regions generated at a single target site, the angle of each damage region with respect to the surface of the skin, the angle between damage regions as viewed from above, etc.

In certain exemplary embodiments of the present invention, the number of individual damage regions generated at a single target site may be defined or limited by the maximum amount of damage desired at the target site on the surface of the skin. By generating multiple regions of damage at a single target site, damage at the target site can be increased, although the degree of local thermal damage can be limited by, e.g., increasing the time duration between successive EMR pulses or by superficially cooling the surface of the skin at the target site.

According to a further exemplary embodiment of the present invention as illustrated in FIG. 5, a cone-shaped region of damage 510 may be created, with a vertex of the cone located at the target site 310 using the exemplary system, process and apparatus of the present invention. This cone-shaped damage pattern 510 may be generated, e.g., by rotating a continuous EMR beam 520 around an axis 530 in a direction indicated by arrow 540, where the beam 520 may form a constant (or varying) angle 550 with respect to the axis 530, and is continuously directed toward the target site 310. The cone-shaped damage pattern 510 can result in a relatively large volume of damaged tissue beneath the skin 140 that can be produced with a small amount of damage to the epidermis 110 at the target site 310. Alternatively, an asymmetrical cone-shaped region of damage may be created by varying the angle of the axis 530 with respect to the surface of the skin 140. Further, two or more cone-shaped damage regions may also be produced beneath a single target site 130 by varying the angle 550 used to generate each such damage region.

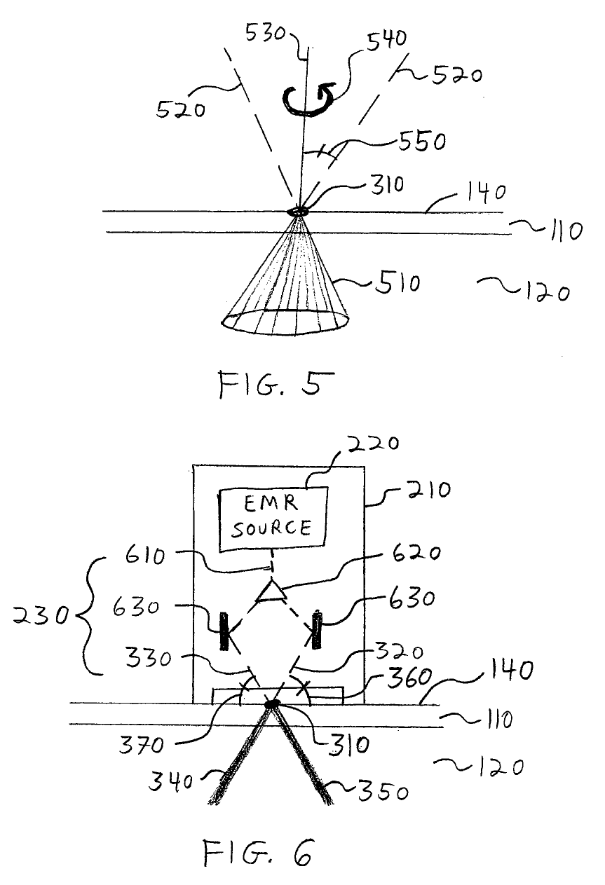

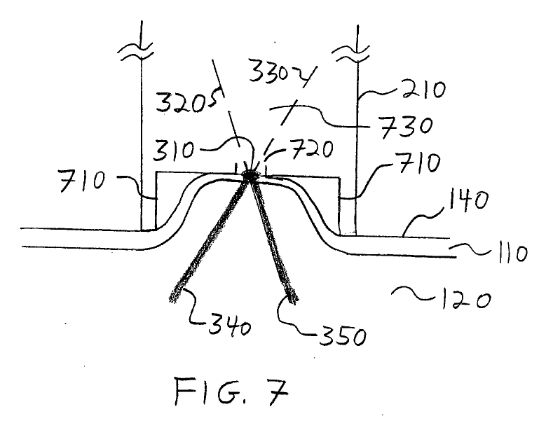

Details of an optical system and arrangement that may implement certain further exemplary embodiments of the present invention are illustrated in FIG. 6. For example, the EMR source 220 can produce an EMR beam 610 within the housing 210. The beam 610 may be directed toward a beam splitter 620, which can separate the beam 610 into the beams 320, 330, with each having approximately half the intensity of the EMR beam 610. The beams 320, 330 can be directed toward mirrors 630, which can be configured to direct the beams 330, 340 toward the target site 310. In this manner, the beams 320, 330 can form the damage regions 340, 350.

Several variations of this exemplary arrangement may be employed. For example, the optical arrangement 230 may be configured such that the beam splitter 620 and/or the mirrors 630 can be translated with respect to the housing 210 and/or each other to vary the angles 360, 370 formed between the beams 320, 330 and the skin surface 140. These exemplary components may be configured such that angles 360 and 370 are approximately equal or are different.

The EMR source 220 may also include two or more lasers or other generators of EMR. The optical arrangement 230 may then be configured to direct two or more EMR beams provided by the two or more lasers or generators of EMR towards a common target site 310 to form desired damage patterns. The two or more EMR beams may be directed toward the skin tissue simultaneously or consecutively.

Alternatively and/or in addition, the mirrors 630 can be shaped to form a cylinder or other continuous shape around an axis formed by the EMR beam 610. Using this exemplary configuration, the beam splitter 620 can be rotated to direct the EMR pulses at various angles around the axis to produce damage patterns similar to those shown in FIGS. 4A and 4B. A continuous wave (“CW”) beam may also be used with this exemplary configuration to form a cone-shaped region of damage such similar to one illustrated in FIG. 5. Alternatively, a portion of such a cone-shaped region of damage may be formed using a CW beam.

A mirror that is configured to rotate and/or otherwise change spatial orientation relative to the beam 610 may be substituted for the beam splitter 620. Using the mirror instead of the beam splitter, a single beam directed at the target site 310 can be provided that has approximately the same intensity as the original EMR beam 610. Additionally, any of these modes and/or arrangements can be combined to produce more complex patterns of damage within the skin tissue, including line-shaped damage regions and/or cone-shaped damage regions oriented at varying angles with respect to the skin surface 140 and/or each other. The characteristics of the EMR beam 610 may also be varied to produce regions of damage at the common target site 310 that may vary with respect to penetration depth, beam width, etc. Alternatively, a beam guide may be used instead of or in addition to the beam splitter 620, where the beam guide may be configured to be movable relative to the housing to vary the direction of the EMR beam 610. The movable beam guide may be used with or without the mirrors 630.

In a further exemplary embodiment of the present invention, as illustrated in FIG. 7, a vacuum-based housing arrangement can be provided to more accurately align the EMR beams with the target site located on the surface of the skin. For example, a recessed chamber 710 containing an orifice 720 can be formed at the lower portion of the housing 210. The housing 210 can be placed over the target site 310, and a vacuum in the region 730 above the orifice 720 can be produced. This vacuum can draw the skin surface 140 upwards into the chamber 710 until the skin surface 140 contacts the upper surface of the chamber 710. The housing 210 can be positioned so that the target site 310 may be located directly beneath the orifice 720. In this manner, the vacuum can maintain the skin surface 140 firmly against the chamber 710 to maintain a more precise alignment of the EMR beams 320, 330 with the target site 310.

Another exemplary advantage of the exemplary embodiment of the present invention illustrated in FIG. 7 is that the effective area of the target site 310 which is damaged during generation of the damage regions 340, 350 within the skin tissue can be reduced. For example, when the skin surface 140 is pulled up into the chamber 710 by the vacuum, the epidermis 110 at the target site 310 may be stretched. This stretching can persist while the EMR beams 320, 330 contact the target site, and penetrate into the epidermis 110 and the dermis 120, thus generating the thermal damage regions 340, 350. After the vacuum is released, the skin surface 140 can relax, and regain its original shape. This relaxation at the skin surface would cause the thermally damaged target site to shrink, and thus can reduce the apparent size of the damaged skin surface at the target area 310. Accordingly, the post-treatment appearance of the skin, can be improved and healing of the epidermis can be more rapid. In addition, the creation of larger regions of damage within the skin tissue can be facilitated while maintaining a relatively smaller region of damage at the surface of the skin.

In still further exemplary embodiments of the present invention, patterns of target sites may be treated over a particular area of skin. Each target site can include several regions of damage associated therewith, as described above. In this manner, a large number of controlled thermal damage patterns can be produced within the skin tissue while generating only a relatively small amount of associated epidermal damage on the surface at the individual target sites.

In other exemplary embodiments of the present invention, the exemplary apparatus 200 illustrated in FIG. 2 may be translated over the skin surface 140, while damage patterns are being generated. In this manner, the target sites may have the form of continuous lines (with a continuous EMR beam) or interrupted/dashed lines or elongated spots (with a pulsed EMR beam). The translation of the system/apparatus 200 can be performed to generate thermal damage over a wider region of the skin in a reasonable amount of time. The system/apparatus 200 may also include a motion detector configured to communicate with the control module 240. The motion detector can include a mechanical sensor, a diode sensor, or the like that is capable of sensing the speed and/or direction of the housing 210 relative to the skin surface 140. The control module 240 can be configured to receive data from the motion sensor, and vary the parameters associated with the EMR source 210 and/or the optical arrangement 230 to produce a variety of thermal damage patterns.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

It will thus be appreciated that those skilled in the art will be able to devise numerous systems, arrangements and methods which, although not explicitly shown or described herein, embody the principles of the invention and are thus within the spirit and scope of the present invention. In addition, all publications, patents and patent applications referenced herein are incorporated herein by reference in their entireties.

What is claimed is:

1. An apparatus, comprising:
a first arrangement configured to provide at least one first electromagnetic radiation and at least one second electromagnetic radiation; and
a second arrangement configured to:
direct the at least one first electromagnetic radiation onto at least one target location on a surface of skin and into at least one first region of tissue below the surface, and
direct the at least one second electromagnetic radiation onto the at least one target location and into a second region of tissue beneath the surface,
wherein the at least one second region is provided at a location that is different from a location of at least a section of the at least one first region.

2. The apparatus according to claim 1, wherein the at least one second region is provided at a location that is substantially different from a location of at least a portion of the at least one first region.

3. The apparatus according to claim 1, wherein the second arrangement comprises an optical arrangement.

4. The apparatus according to claim 3, wherein at least one of the at least one first electromagnetic radiation or the at least one second electromagnetic radiation is an ablative laser beam.

5. The apparatus according to claim 4, wherein the ablative laser beam is provided by at least one of a CO2 laser or an ER:YAG laser.

6. The apparatus according to claim 1, wherein at least one of the at least one first electromagnetic radiation or the at least one second electromagnetic radiation is a non-ablative radiation.

7. The apparatus according to claim 6, wherein the non-ablative radiation is provided by at least one of a laser, a flashlamp, a tungsten lamp, a diode or a diode array.

8. The apparatus according to claim 3, wherein the optical arrangement comprises a beam splitter.

9. The apparatus according to claim 3, wherein the optical arrangement comprises at least one of a mirror or a prism beam splitter.

10. The apparatus according to claim 1, further comprising a vacuum arrangement configured to pull the target location closer to the second arrangement.

11. The apparatus according to claim 1, wherein the second arrangement is configured to direct the at least one first electromagnetic radiation at a first angle with respect to the surface of the skin, and is further configured to direct the at least one second electromagnetic radiation at a second angle with respect to the surface of the skin, and wherein the first angle is different from the second angle.

12. The apparatus according to claim 1, wherein the second arrangement is configured to rotate a direction of at least one of the at least one first electromagnetic radiation and the at least one second electromagnetic radiation around an axis which passes through the target location.

13. The apparatus according to claim 1, wherein at least one of the at least one first electromagnetic radiation and the at least one second electromagnetic radiation is provided in a form of at least one pulse.

14. The apparatus according to claim 1, wherein the at least one first electromagnetic radiation and the at least one second electromagnetic radiation are provided in a single continuous wave beam of radiation.

15. The apparatus according to claim 14, wherein the second arrangement is configured to direct the continuous wave beam of radiation at a plurality of angles with respect to the surface of the skin.

16. The apparatus according to claim 14, wherein the second arrangement is configured to rotate the continuous wave beam of radiation around an axis which passes through the target location.

17. A system for treating dermatological conditions, comprising:
a first arrangement configured to provide at least one first electromagnetic radiation and at least one second electromagnetic radiation; and
a second arrangement configured to:
direct the at least one first electromagnetic radiation onto at least one target location on a surface of skin and into at least one first region of tissue below the surface, and
direct the at least one second electromagnetic radiation onto the at least one target location and into a second region of tissue beneath the surface; and
a controller arrangement configured to at least one of select or control a plurality of parameters associated with at least one of the at least one first electromagnetic radiation or at least one second electromagnetic radiation, and which is further configured to control the second arrangement,
wherein the at least one second region is provided at a location that is different from a location of at least a section of the at least one first region.

18. The apparatus according to claim 17, wherein the second arrangement comprises an optical arrangement.

19. A method for treating dermatological conditions, comprising:
directing at least one first electromagnetic radiation onto at least one target location on a surface of skin and into at least one first region of tissue below the surface, and
directing at least one second electromagnetic radiation onto the at least one target location and into a second region of tissue beneath the surface,
wherein the at least one second region is provided at a location that is different from a location of at least a section of the at least one first region.

20. The method according to claim 19, wherein at least one of the at least one first electromagnetic radiation and the at least one second electromagnetic radiation is provided in a form of at least one pulse.
